(19) United States
(12) Patent Application Publication
(10) Pub. No.: US 2022/0310268 A1
(43) Pub. Date: Sep. 29, 2022

(54) SYSTEM AND METHOD FOR USE OF PATTERN DETECTION IN ASSESSING SELF-REPORTED SYMPTOM DATA

(71) Applicant: ORACLE INTERNATIONAL CORPORATION, Redwood Shores, CA (US)

(72) Inventors: Saurabh Verma, Cupertino, CA (US); Shailendra Mishra, Fremont, CA (US); Rebecca Laborde, Byron, MN (US)

(21) Appl. No.: 17/214,659

(22) Filed: Mar. 26, 2021

(51) Int. Cl. G16H 50/70; G06F 16/245; G06F 16/23; G16H 10/20; G16H 10/60; G16H 40/67; G16H 40/20

(52) U.S. Cl. CPC G16H 50/70; G06F 16/245; G06F 16/2358; G16H 10/20; G16H 10/60; G16H 40/67; G16H 40/20; G16H 50/80

(57) ABSTRACT

In accordance with an embodiment, described herein are systems and methods for use of data analytics in medical applications, including the use of pattern detection in assessing self- reported participant symptom data indicative of possible illness. A patient monitoring system or service can be provided, for example at an analytics cloud environment. The system is adapted to capture self-reported participant symptom data from individual participants, and track changes in their reported symptoms over time. The system performs data queries against the received participant symptom data, to identify patterns in the data indicative of participant clusters and episodes indicative of possible illness, which information can then be provided, for example, to a medical organization system and used to respond to investigative queries. The approach can accommodate voluntary and/or intermittent reporting, including sparsity or gaps in the input stream of symptom data received from the participants.

### TECHNICAL FIELD

Embodiments described herein are generally related to the use of data analytics in medical applications, and to systems and methods for patient monitoring, including use of pattern detection in assessing self-reported participant symptom data indicative of possible illnesses.

### BACKGROUND

Today's medical health systems maintain databases of symptom data which can be useful in determining trends within the data. However, in situations where patient participation or symptom reporting may be voluntary and/or intermittent, the recorded symptom data may be sparse, and gaps in the data can impact its use in various investigations.

For example, when participation is voluntary, there may be no requirement that a person exhibiting symptoms, or who has been tested for the presence of a particular medical condition, will actually return to report any change in their symptoms, which information might be useful in treatment of their condition, or tracing within the wider community. Voluntary and/or intermittent reporting may also impact the ability to accurately assess a particular patient's progress over a period of time.

### SUMMARY

In accordance with an embodiment, described herein are systems and methods for use of data analytics in medical applications, including the use of pattern detection in assessing self-reported participant symptom data indicative of possible illness. A patient monitoring system or service can be provided, for example at an analytics cloud environment. The system is adapted to capture self-reported participant symptom data from individual participants, and track changes in their reported symptoms over time. The system performs data queries against the received participant symptom data, to identify patterns in the data indicative of participant clusters and episodes indicative of possible illness, which information can then be provided, for example, to a medical organization system and used to respond to investigative queries. The approach can accommodate voluntary and/or intermittent reporting, including sparsity or gaps in the input stream of symptom data received from the participants.

### BRIEF DESCRIPTION OF THE DRAWINGS

FIG. 1 illustrates an example analytics cloud environment, in accordance with an embodiment.

FIG. 2 illustrates the use of an analytics cloud environment to provide a patient monitoring system or service, in accordance with an embodiment.

FIG. 3 illustrates the use of patient monitoring system or service, in accordance with an embodiment.

FIG. 4 further illustrates the use of patient monitoring system or service, in accordance with an embodiment.

FIG. 5 further illustrates the use of patient monitoring system or service, in accordance with an embodiment.

FIG. 6 further illustrates the use of patient monitoring system or service, in accordance with an embodiment.

FIG. 7 further illustrates the use of patient monitoring system or service, in accordance with an embodiment.

FIG. 8 further illustrates the use of patient monitoring system or service, in accordance with an embodiment.

FIG. 9 illustrates the use of a patient monitoring system or service, and pattern detection in assessing self-reported participant symptom data, to determine participant clusters, in accordance with an embodiment.

FIG. 10 further illustrates the use of a patient monitoring system or service, and pattern detection in assessing self-reported participant symptom data, to determine episodes indicative of possible illness, in accordance with an embodiment.

FIG. 11 further illustrates the use of a patient monitoring system or service to determine participant clusters and episodes indicative of possible illness, in accordance with an embodiment.

FIG. 12 illustrates an example dashboard, in accordance with an embodiment.

FIG. 13 illustrates another example dashboard, in accordance with an embodiment.

FIG. 14 illustrates another example dashboard, in accordance with an embodiment.

FIG. 15 illustrates an example method or process for providing a patient monitoring system or service, including support for use of pattern detection in assessing self-reported participant symptom data, in accordance with an embodiment.

### DETAILED DESCRIPTION

As described above, today's medical health systems maintain databases of symptom data which can be useful in determining trends within the data. However, in situations where patient participation or symptom reporting may be voluntary and/or intermittent, the recorded symptom data may be sparse, and gaps in the data can impact its use in various investigations.

In accordance with an embodiment, described herein are systems and methods for use of data analytics in medical applications, including the use of pattern detection in assessing self-reported participant symptom data indicative of possible illness.

In accordance with an embodiment, a patient monitoring system or service can be provided, for example at an analytics cloud environment. The system is adapted to capture self-reported participant symptom data from individual participants, and track changes in their reported symptoms over time. The system performs data queries against the received participant symptom data, to identify patterns in the data indicative of participant clusters and episodes indicative of possible illness, which information can then be provided, for example, to a medical organization system and used to respond to investigative queries.

In accordance with an embodiment, the approach can accommodate voluntary and/or intermittent reporting, including sparsity or gaps in the input stream of symptom data received from the participants.

For example, in accordance with an embodiment, the systems and methods described herein can be used to capture self-reported participant symptom data provided by individual participants who are either suspected to have contracted, or may have been potentially exposed to, severe acute respiratory syndrome coronavirus disease (e.g., Coronavirus Disease 2019, COVID-19, COVID), and track changes in their reported symptoms over time. Such information can then be used to identify patterns in the participant symptom data indicative of participant clusters, and episodes indicative of possible coronavirus-related illnesses.

In accordance with an embodiment, the determination of participant clusters can be used, for example, to identify continuous sets of symptom reports; or segment a population into irregular reporters versus regular reporters, and correlate their reports to actual symptoms. An identification of such participant clusters is useful in aggregating participant behavioral information at a cluster level, and correlating the information provided with actual symptoms and symptom density.

In accordance with an embodiment, the determination of episodes can be used, for example, to identify actual bouts of illness/sickness reflected in the fine-grain participant symptom data stream, and aggregate and report various parameters regarding those bouts of illness/sickness, for further classification. An identification of such episodes is useful in summarizing disease characteristics at a higher level which can be used, for example, to analyze the emergence of possible COVID infections or trends within the community.

### Analytics Cloud Environment

In accordance with an embodiment, a patient monitoring system or service can be provided at an analytics cloud environment, for example as described below, for use of data analytics in medical applications, including the use of pattern detection in assessing self-reported participant symptom data indicative of possible illness.

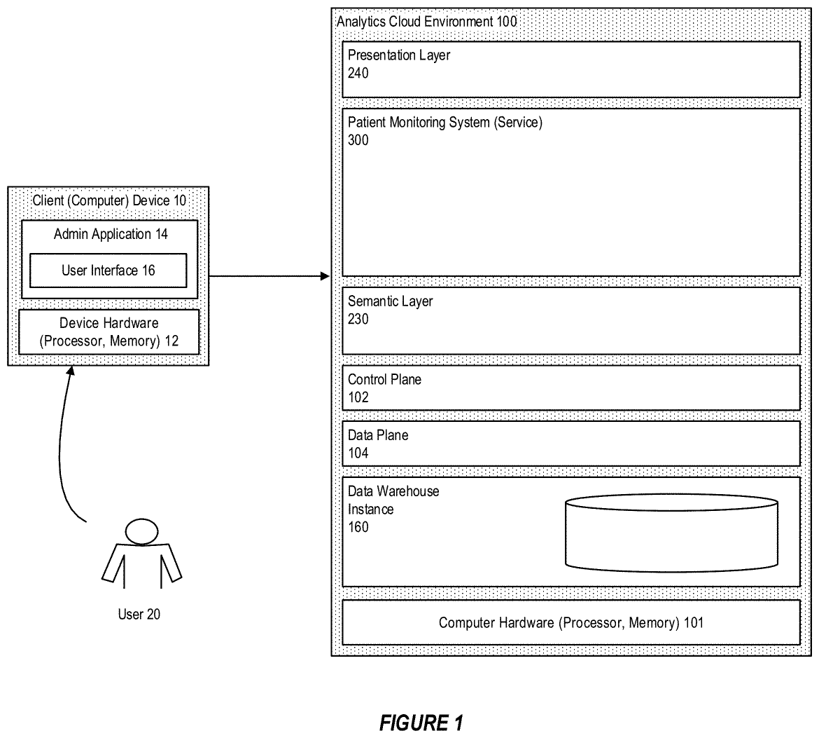

FIG. 1 illustrates an example analytics cloud environment, in accordance with an embodiment.

In accordance with an embodiment, the example analytics cloud environment illustrated in FIG. 1 is provided for purposes of illustrating an example of a data analytics environment that can provide a patient monitoring system or service as described herein. Examples of various types of analytics cloud environments include Oracle Analytics Cloud, Oracle Analytics for Applications, or Oracle Fusion Analytics. In accordance with various embodiments, the patient monitoring system or service described herein can alternatively be provided by other types of computing environment.

As illustrated in FIG. 1, in accordance with an embodiment, the analytics cloud environment 100 can be provided by, or otherwise operate at, a computer system having a computer hardware (e.g., processor, memory) 101, and including one or more software components operating as a control plane 102, and a data plane 104, and providing access to a data warehouse, or data warehouse instance 160.

In accordance with an embodiment, the components and processes illustrated in FIG. 1, and as further described herein with regard to various other embodiments, can be provided as software or program code executable by a computer system or other type of processing device. For example, the components and processes described herein can be provided by a cloud computing system, or other suitably-programmed computer system.

In accordance with an embodiment, the control plane enables access by a client computer device 10 having a device hardware 12, administrative application 14, and user interface 16, under control of a user (tenant) 20 and/or a cloud environment having a provisioning component.

In accordance with an embodiment, the data plane can include a data pipeline or process layer and a data transformation layer that together process operational or transactional data from an organization's enterprise software application or data environment. In accordance with an embodiment, the data transformation layer can include a data model, such as, for example, a knowledge model (KM), or other type of data model, that the system uses to transform the transactional data, into a model format understood by the analytics cloud environment. The model format can be provided in any data format suited for storage in a data warehouse.

In accordance with an embodiment, a data pipeline or process can be scheduled to execute at intervals (e.g., hourly/daily/weekly) to extract transactional data from a software application or database environment.

In accordance with an embodiment, an extract process can extract the transactional data, whereupon extraction the data pipeline or process can insert extracted data into a data staging area, which can act as a temporary staging area for the extracted data. The data quality component and data protection component can be used to ensure the integrity of the extracted data. For example, the data quality component can perform validations on the extracted data while the data is temporarily held in the data staging area. In accordance with an embodiment, when the extract process has completed its extraction, the data transformation layer can be used to begin the transform process, to transform and load the extracted data into the data warehouse.

### Patient Monitoring System (Service)

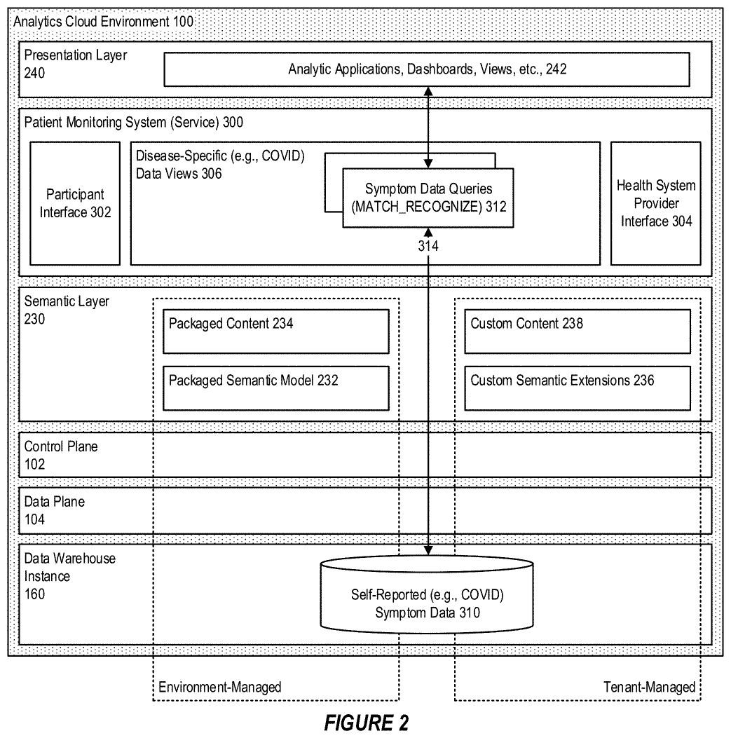

FIG. 2 illustrates the use of an analytics cloud environment to provide a patient monitoring system or service, in accordance with an embodiment.

As illustrated in FIG. 2, in accordance with an embodiment, an analytics cloud environment can include a semantic layer 230, packaged (out-of-the-box, initial) semantic model 232 that provides a packaged content 234, and semantic extensions 236 that extend the packaged semantic model and provide custom content 238 to the presentation layer 240.

In accordance with an embodiment, the presentation layer enables access to a data warehouse instance, database, or data content using, for example, a software analytic application, dashboard, views 242, or other type of report or interface as may be provided by products such as, for example, Oracle Analytics Cloud, Oracle Analytics for Applications, or Oracle Fusion analytics.

As further illustrated in FIG. 2, in accordance with an embodiment, a patient monitoring system, or service 300 can be provided, which includes a participant interface 302, a provider interface 304, and one or more disease-specific data views 306.

In accordance with an embodiment, the participant interface enables communication with mobile devices or other devices that include a participant reporting application and that are adapted receive from participants a symptom data, for example via an email or text survey communicated by the patient monitoring system or service to the participant's device for completion by the participant.

In accordance with an embodiment, the provider interface enables communication with medical organization systems or other devices that enable display of a dashboard of information that allows the organization or user to perform interactive reporting or visualizations associated with the participant symptom data.

In accordance with an embodiment, the disease-specific data views, enable data queries to be performed on the data and used to create visualizations. The result of such data queries can be provided directly to a medical organization system via the provider interface, or can be provided via and/or combined with information provided by the presentation layer.

As further illustrated in FIG. 2, in accordance with an embodiment, the patient monitoring system, can access information stored in a self-reported (e.g., COVID) symptom database 310.

For example, in accordance with an embodiment, the information saved in the database can be stored in a data warehouse instance which is populated using any of the above means.

In accordance with an embodiment, the system is adapted to capture self-reported participant symptom data from individual participants, and track changes in their reported symptoms over time. The system performs data queries 312 against the received participant symptom data, to identify patterns 314 in the data indicative of participant clusters and episodes indicative of possible illness, which information can then be provided, for example, to a medical organization system and used to respond to investigative queries.

In accordance with an embodiment, the patient monitoring system or service as described herein can use pattern-matching capabilities supported by a database that stores the participant symptom data, such as for example the use of a MATCH_RECOGNIZE data query, to identify and abstract the above participant and episode concepts from fine-grained participant symptom data. Such pattern-matching data queries can be used to identify a particular shape in a stream of data, and to sift through the fine-grained records and identify participant clusters and episodes.

For example, in accordance with an embodiment, the patient monitoring system or service can provide a disease-specific data view into the reported participant symptom data, which utilizes a MATCH_RECOGNIZE data query to assess, within a 3-day rolling window, various, e.g., COVID-related symptoms associated with one or more patient participants; including filtering or grouping symptom reports and tracking them over time.

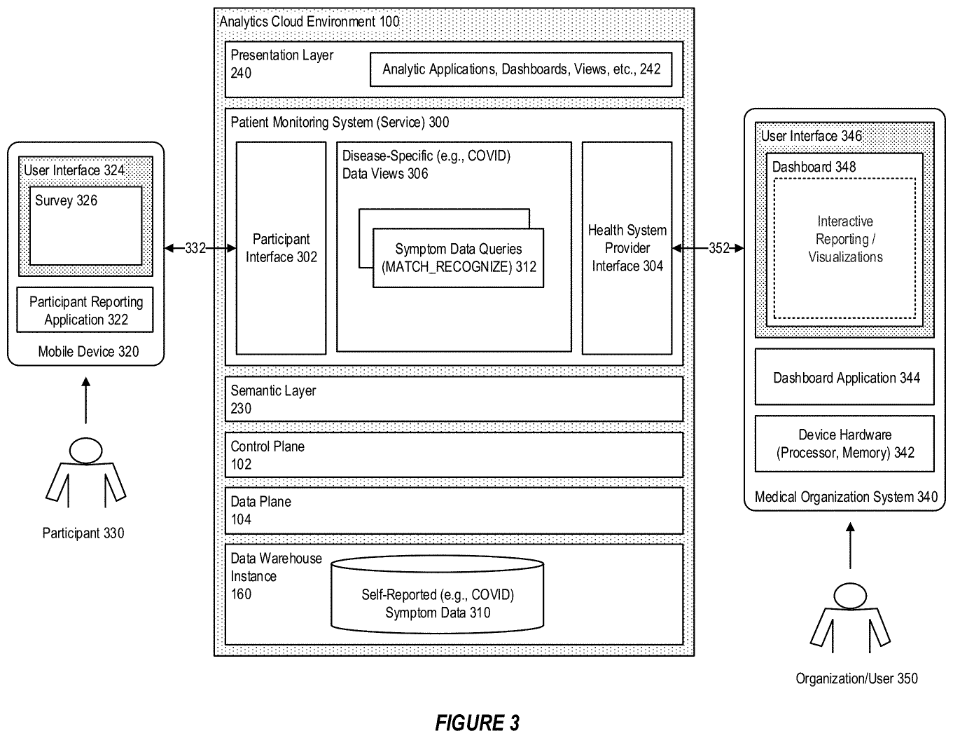

FIG. 3 illustrates the use of patient monitoring system or service, in accordance with an embodiment.

As illustrated in FIG. 3, in accordance with an embodiment, the system can receive participant symptom data from a participant via a mobile device 320 that includes participant reporting application 322, user interface 324, which provides displays or otherwise provides access to a survey 326, which the participant 330 can complete, to provide their participant symptom data.

In accordance with an embodiment, the participant device can communicate 332 with the participant interface of the system, via for example the Internet or other communication means to provide self-reported participant symptom data.

As further illustrated in FIG. 3 in accordance with an embodiment, each organization can access the results of the surveys using a medical organization system 340, having a device hardware 342, dashboard application 344, and user interface 346 that enables display of a dashboard 348 of information that allows the organization or user 350 to perform interactive reporting or visualizations.

In accordance with an embodiment, the medical organization system can communicate 352 with the provider interface of the system, via for example the Internet or other communication means of receiving or accessing the self-reported participant symptom data.

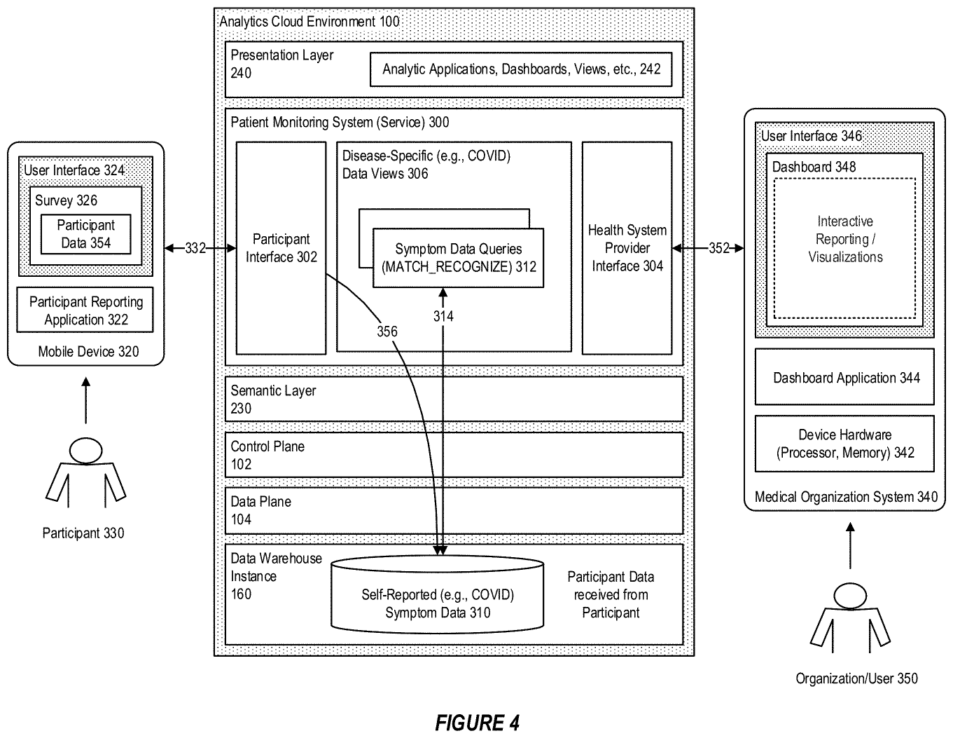

FIG. 4 further illustrates the use of patient monitoring system or service, in accordance with an embodiment.

As illustrated in FIG. 4, in accordance with an embodiment, as participant provide survey information the participant data 354 is stored 356 in the database for subsequent use in performing the data queries.

### Pattern-Matching with MATCH_RECOGNIZE

In accordance with an embodiment, the patient monitoring system or service as described herein can use pattern-matching capabilities such as for example the use of a MATCH_RECOGNIZE data query, to identify and abstract the above participant and episode concepts from fine-grained participant symptom data. For example, MATCH_RECOGNIZE enables the system to perform data queries to accomplish tasks such as, for example:

Logically partition and order the data that is used in the MATCH_RECOGNIZE clause with PARTITION BY and ORDER BY clauses.

Define patterns of rows to seek using a PATTERN clause. These patterns can use a regular expression syntax applied to the pattern variables.

Specify the logical conditions required to map a row to a row pattern variable in a DEFINE clause. DEFINE indicates the conditions that must be met for a row to map to a row pattern variables STRT, DOWN, and UP. Because there is no condition for STRT, any row can be mapped to STRT.

Define measures, which are expressions usable in other parts of the SQL query, in a MEASURES clause.

An indication of ONE ROW PER MATCH means that for every pattern-match found, there will be one row of output.

An indication of AFTER MATCH SKIP TO LAST UP means that whenever a match is found, the search will be restarted at the row that is the last row of the UP pattern variable.

An indication of PATTERN (STRT DOWN+ UP+) indicates that the pattern being searched has three pattern variables: STRT, DOWN, and UP. The plus sign (+) after DOWN and UP means that at least one row must be mapped to each of them.

For example, when applied to a set of data, the MATCH_RECOGNIZE clause performs these steps:

The row pattern input table is partitioned according to the PARTITION BY clause. Each partition consists of the set of rows of the input table that have the same value on the partitioning columns.

Each row pattern partition is ordered according to the ORDER BY clause.

Each ordered row pattern partition is searched for matches to the PATTERN.

Pattern-matching operates by seeking the match at the earliest row, considering the rows in a row pattern partition in the order specified by the ORDER BY clause.

Pattern-matching in a sequence of rows is an incremental process, with one row after another examined to see if it fits the pattern. If no match is found at the earliest row, the search moves to the next row in the partition, checking if a match can be found starting with that row. After a match is found, row pattern-matching calculates the row pattern measure columns, which are expressions defined by the MEASURES clause.

Using ONE ROW PER MATCH, pattern-matching generates one row for each match that is found. If ALL ROWS PER MATCH is used, every row that is matched is included in the pattern-match output.

The AFTER MATCH SKIP clause determines where row pattern-matching resumes within a row pattern partition after a non-empty match is found.

The above is provided by way of example, to illustrate the manner by which a MATCH_RECOGNIZE data query or clauses can be used to access a data warehouse or database that stores self-reported participant symptom data and provide resultant information to the presentation layer and/or to the health system provider interface. In accordance with other embodiments, other types of data queries or clauses can be used to perform pattern recognition.

### Patient Monitoring

In accordance with an embodiment, the system is adapted to capture self-reported symptom data from individual participants, and track changes in their reported symptoms over time.

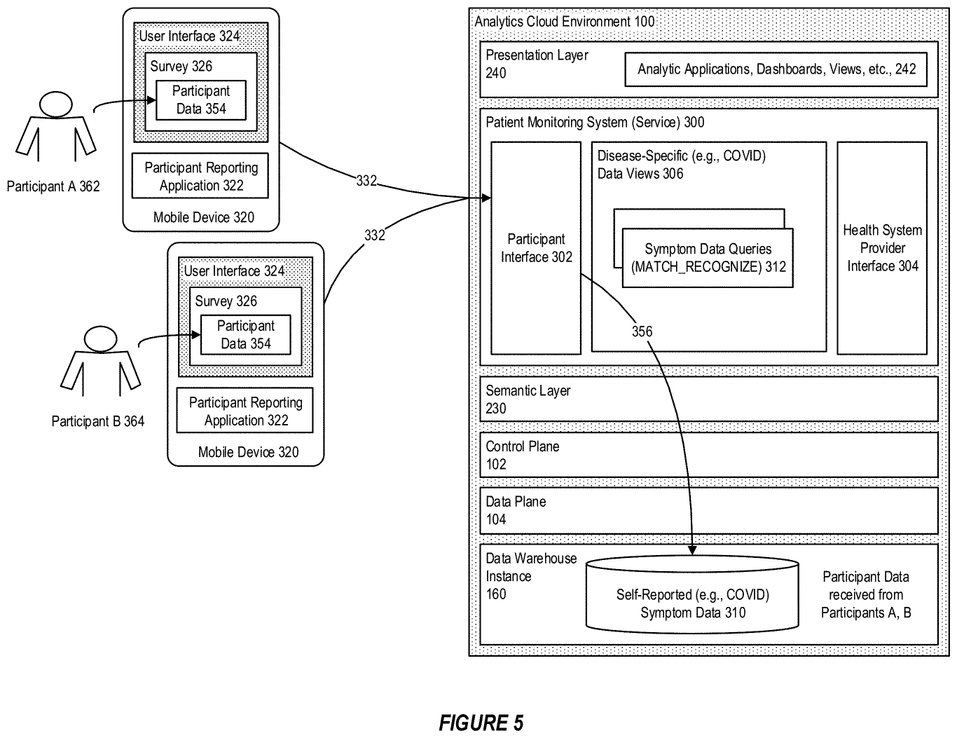

FIG. 5 further illustrates the use of patient monitoring system or service, in accordance with an embodiment.

As illustrated in FIG. 5, in accordance with an embodiment, at a particular point in time, each of a plurality of participants, in this example participant A 362 and participant B 364, can respond to a survey provided by an organization, and their participant data received from participants A and B recorded in the self-reported symptom database.

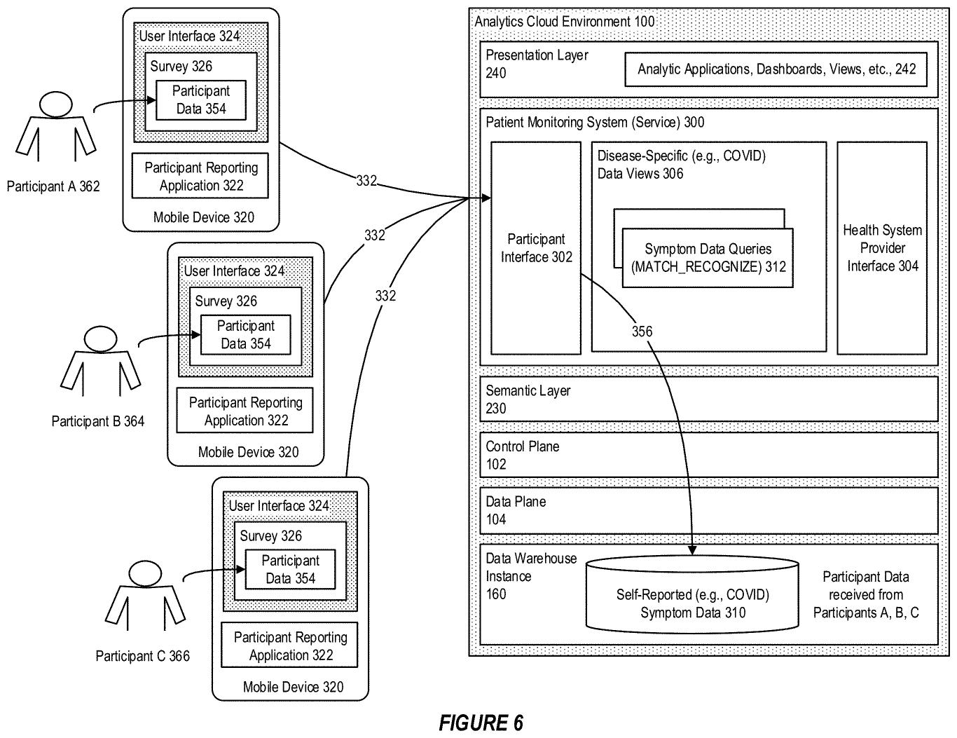

FIG. 6 further illustrates the use of patient monitoring system or service, in accordance with an embodiment.

As illustrated in FIG. 6, in accordance with an embodiment, at a subsequent point in time, each of the plurality of participants A, B, and in this example an additional participant C 366, can respond to a survey, and their participant data received from participants A, B, C recorded in the self-reported symptom database.

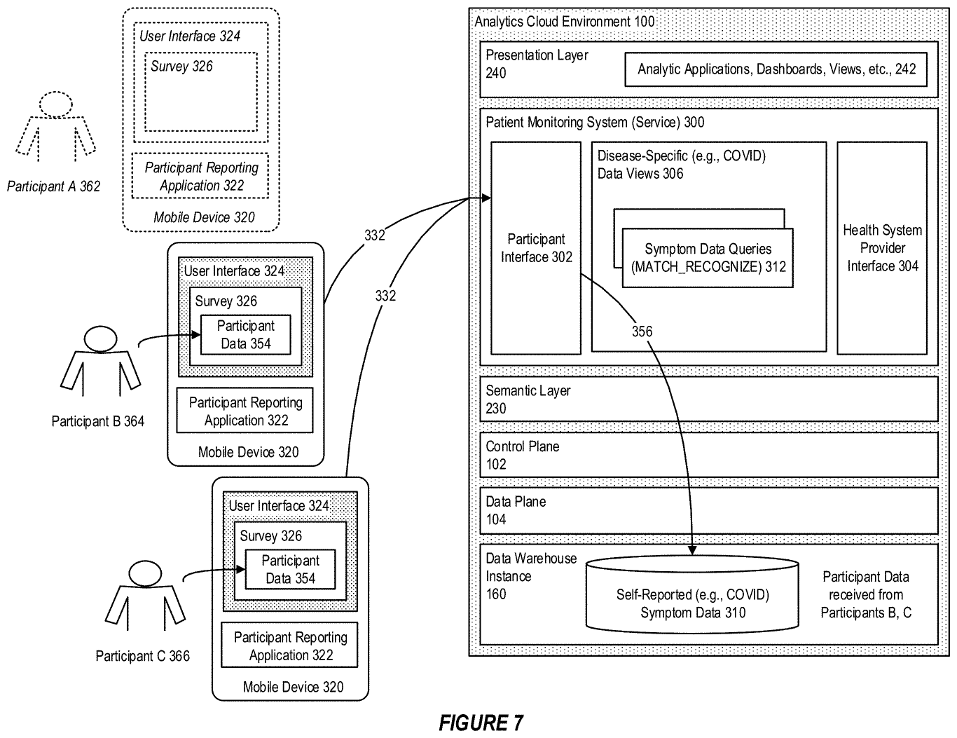

FIG. 7 further illustrates the use of patient monitoring system or service, in accordance with an embodiment.

As illustrated in FIG. 7, in accordance with an embodiment, at a subsequent point in time, participant A may not respond to a survey or otherwise report their symptoms; however each of the remaining participants B, C have responded to a survey, and their participant data received from participants B, C (but not A) recorded in the self-reported symptom database.

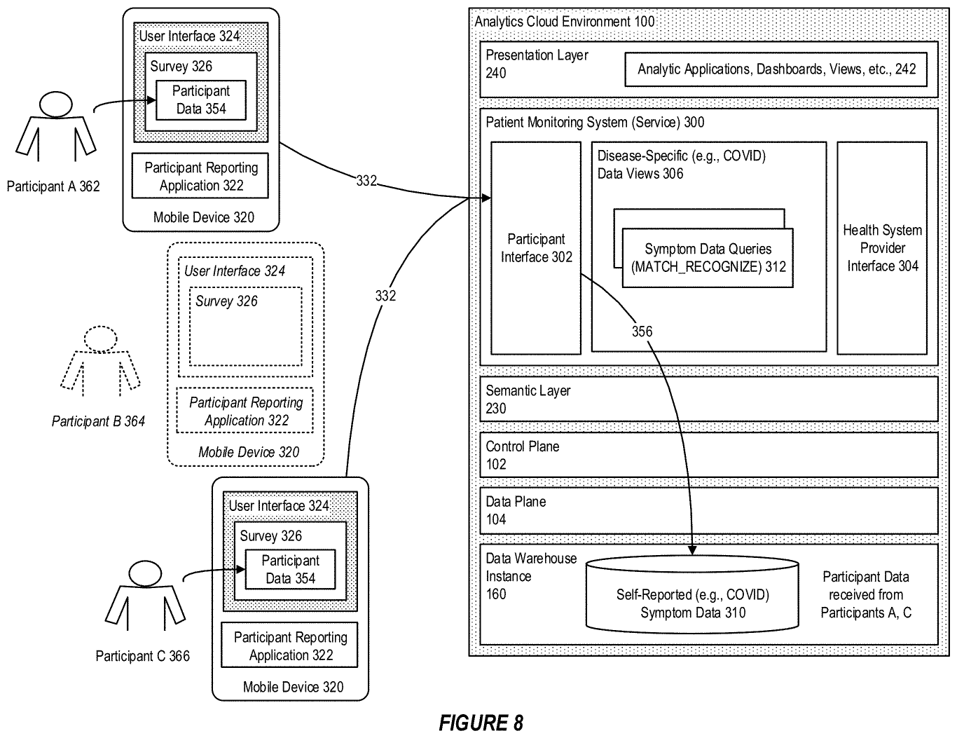

FIG. 8 further illustrates the use of patient monitoring system or service, in accordance with an embodiment.

As illustrated in FIG. 8, in accordance with an embodiment, at a subsequent point in time, participant B may not respond to a survey or otherwise report their symptoms; however each of the remaining participants A, C have responded to a survey, and their participant data received from participants A, C (but not B) recorded in the self-reported symptom database.

As illustrated in FIGS. 5-8, in situations where patient participation or symptom reporting may be voluntary and/or intermittent, the recorded symptom data may be sparse. For example, when participation is voluntary, there may be no requirement that a person exhibiting symptoms, or who has been tested for the presence of a particular medical condition, will actually return to report any change in their symptoms.

### Determination of Participant Clusters

In accordance with an embodiment, the system performs data queries against the received participant symptom data, to identify patterns in the data indicative of participant clusters and episodes indicative of possible illness, which information can then be provided, for example, to a medical organization system and used to respond to investigative queries.

For example, in accordance with an embodiment, the U.S. Centers for Disease Control and Prevention (CDC) may provide a “COVID-like illness” symptom definition as including “Fever”=Yes AND (“Cough”=Yes OR “Shortness of Breath”=Yes OR “Gasping for Air”=Yes OR “Difficult Breathing”=Yes); for 3 consecutive days OR at least 2 out of 3 days with the middle day missing. In accordance with this definition, if data for the middle day is present, but does not have that combination of symptoms, then the present day should not be marked as COVID-like illness.

In accordance with an embodiment, the patient monitoring system or service can provide a disease-specific data view into the reported participant symptom data, which utilizes a MATCH_RECOGNIZE data query to assess, within a 3-day rolling window, various, e.g., COVID-related symptoms associated with one or more patient participants; including filtering or grouping symptom reports and tracking them over time.

Such tracking can be discontinued once there is, e.g., a 5-day gap in reported symptoms, again as determined by a MATCH_RECOGNIZE data query. Provided information can be used to assess, for example, whether particular participants may be stopping and/or restarting their reporting of symptoms, or to distinguish situations wherein reported symptoms indicate the presence of perhaps seasonal allergies or influenza versus, e.g., a COVID-like illness.

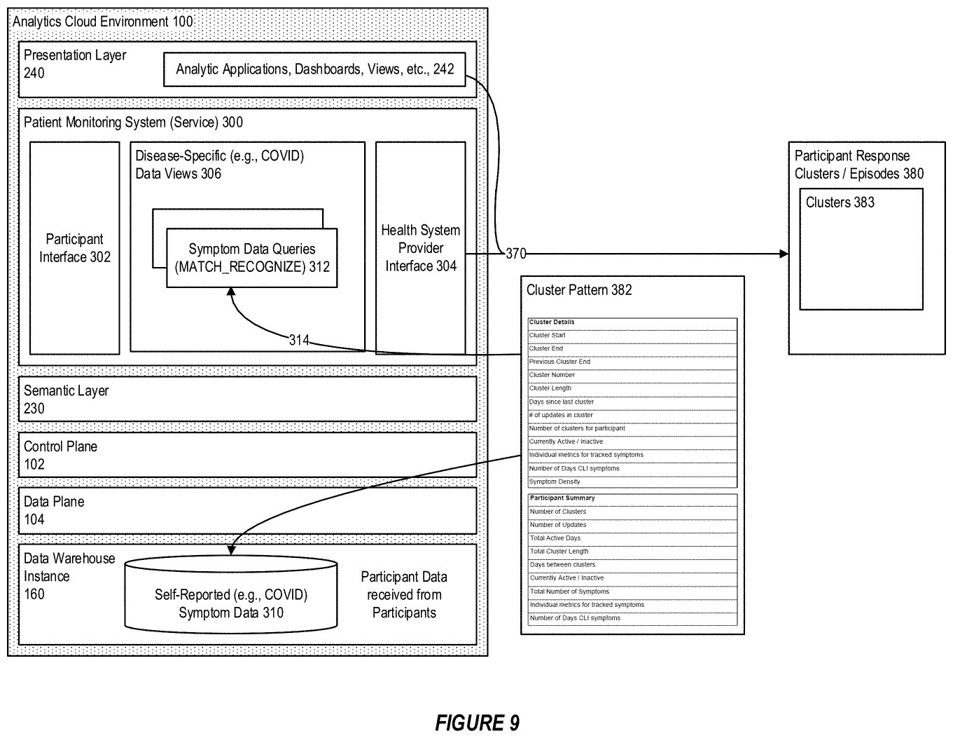

FIG. 9 illustrates the use of a patient monitoring system or service, and pattern detection in assessing self-reported participant symptom data, to determine participant clusters, in accordance with an embodiment.

As illustrated in FIG. 9, in accordance with an embodiment, the patient monitoring system or service is adapted to provide the results of data queries against the received participant symptom data, via the provider interface 370, for example an indication 380 of participant clusters and episodes indicative of possible illness, or other information, to a medical organization system and used to respond to investigative queries.

For example, in accordance with an embodiment, the system can perform data queries against the received participant symptom data, as defined by a cluster pattern 382, to determine participant clusters 383, which information can then be provided, for example, to a medical organization system and used to respond to investigative queries.

For example, as illustrated in FIG. 9 (and in example patterns 1 and 2 below) a cluster pattern can be defined in accordance with CDC or another definition for COVID-like illness, to determine clusters as a group of status reports from a participant with <5 days gap; and for individual metrics for tracked symptoms, including in this example: Fever (FVR); Cough (CGH); Shortness of Breath (SOB); Sore Throat (STH); Diarrhea (DIA); Bluish Lips or Face (BLU); Loss of Taste or Smell, (LTS); Nausea or Vomiting (NAU); Confused Inability to Arouse (CIA); Persistent Pain (PAI); Muscle Pain (MP); Headache (HA); Congestion (CON); Fatigue (FTG); Chills (CH).

Cluster Details
Cluster Start
Cluster End
Previous Cluster End
Cluster Number
Cluster Length
Days since last cluster
# of updates in cluster
Number of clusters for participant
Currently Active/Inactive
Individual metrics for tracked symptoms
Number of Days CLI symptoms
Symptom Density

### EXAMPLE PATTERN 1

### Participant Cluster Details

In accordance with an embodiment, the example symptoms and cluster pattern definition illustrated in FIG. 9, and in example patterns 1 and 2), is provided for purposes of illustration. In accordance with other embodiments, other cluster definitions of symptoms and cluster patterns can be used, for example in accordance with other definitions for COVID-like illness.

Participant Summary
Number of Clusters
Number of Updates
Total Active Days
Total Cluster Length
Days between clusters
Currently Active/Inactive
Total Number of Symptoms
Individual metrics for tracked symptoms
Number of Days CLI symptoms

### EXAMPLE PATTERN 2

### Participant Summary

In accordance with an embodiment, the patient monitoring system or service is adapted to perform SQL data queries that correspond to the defined pattern, including for example one or more pattern-matching SQL data queries that incorporate a MATCH_RECOGNIZE clause, for example as illustrated in example (MATCH_RECOGNIZE) query 1 below, to access the data warehouse or database that stores self-report participant symptom data and provide resultant information to the presentation layer and/or to the health system provider interface.

```
drop materialized view status_updates_COVID_view;
create materialized view status_updates_COVID_view
select
  status_updates_COVID.id, status_updates_COVID.HEALTH_SYSTEM_ID
,EXPOSURE_LOCATION_ID,EXPOSURE_DURATION_ID,EXPOSURE_VICINITY_ID,
  person_id, current_symptoms,people.ethnicity_id, people.facility_id,
  COVID_SYMPTOM_CURRENT_DAY,COVID_SYMPTOM_PREVIOUS_DAY,COVID_SYMPTOM_PREVIOUS_2_DAY,
  FVR_3_DAY_YN,CGH_3_DAY_YN,SOB_3_DAY_YN,STH_3_DAY_YN,LTS_3_DAY_YN,NAU_3_DAY_YN,
DIA_3_DAY_YN,BLU_3_DAY_YN,CIA_3_DAY_YN,PAI_3_DAY_YN,MP_3_DAY_YN,HA_3_DAY_YN,CON_3_DAY_YN,FTG_3
_DAY_YN,CH_3_DAY_YN,
status_updates_COVID.created,status_updates_COVID.created_dt,created_unified_date,prev_date_1,
prev_date_2,
  CASE WHEN COVID_SYMPTOM_CURRENT_DAY =‘Y’ AND COVID_SYMPTOM_PREVIOUS_2_DAY = ‘Y’ AND
COVID_SYMPTOM_PREVIOUS_DAY in (‘Y’,‘NOT_FOUND’) THEN ‘Y’ ELSE ‘N’ END AS COVID_3_DAY_YN,
  FIRST_VALUE(CASE WHEN COVID_SYMPTOM_CURRENT_DAY =‘Y’ AND COVID_SYMPTOM_PREVIOUS_2_DAY = ‘Y’
AND COVID_SYMPTOM_PREVIOUS_DAY in (‘Y’,‘NOT_FOUND’) THEN created_unified_date END) IGNORE
NULLS
  OVER (PARTITION BY person_id ORDER BY created_unified_date asc ROWS BETWEEN UNBOUNDED
PRECEDING AND UNBOUNDED FOLLOWING) AS FIRST_CLI_SYMPTOM_DATE
FROM
    (select
  id, HEALTH_SYSTEM_ID ,EXPOSURE_LOCATION_ID,EXPOSURE_DURATION_ID,EXPOSURE_VICINITY_ID,
  person_id,
  current_symptoms, CASE WHEN instr(current_symptoms,‘FVR’)>0 AND (
instr(current_symptoms,‘CGH’)>0 OR instr(current_symptoms,‘SOB’)>0 OR
instr(current_symptoms,‘GAS’)>0 OR instr(current_symptoms,‘TLK’) >0 ) THEN ‘Y’ ELSE ‘N’ END
  AS COVID_SYMPTOM_CURRENT_DAY ,
** SNIPPED **
;
drop materialized view participant_clusters;
create materialized view participant_clusters
select person as person_id, participant_clusters.HEALTH_SYSTEM_ID,people.ethnicity_id,
people.facility_id,
  cluster_number, cluster_start, cluster_end, cluster_length, NUMBER_of_updates,
NUMBER_of_days_COVID,
  LAG(CLUSTER_END,1,null) OVER (PARTITION by person ORDER BY CLUSTER_NUMBER) as
previous_cluster_end, cluster_start - LAG(CLUSTER_END,1,null)
  OVER (PARTITION by person ORDER BY CLUSTER_NUMBER) as DAYS_SINCE_LAST_CLUSTER, FEVER, COUGH,
SHORTNESS_OF_BREADTH,SORE_THROAT,DIARRHEA,BLUISH_LIPS_OR_FACE,
LOSS_OF_TASTE_SMELL,NAUSEA_OR_VOMITING,CONFUSED_INABILITY_AROUSE,PERSISTENT_PAIN,MUSCLE_PAIN,H
EADACHE,CONGESTION,FATIGUE,CHILLS,
  FEVER+ COUGH+ SHORTNESS_OF_BREADTH+SORE_THROAT+DIARRHEA+BLUISH_LIPS_OR_FACE+
LOSS_OF_TASTE_SMELL+NAUSEA_OR_VOMITING+CONFUSED_INABILITY_AROUSE+PERSISTENT_PAIN+MUSCLE_PAIN+H
EADACHE+CONGESTION+FATIGUE+CHILLS AS TOTAL_SYMPTOMS
from (
  SELECT
  person,HEALTH_SYSTEM_ID,cluster_number, trunc(cluster_start) as cluster_start,
trunc(cluster_end) as cluster_end,
  NUMBER_of_updates, NUMBER_of_days_COVID, cluster_end-cluster_start as cluster_length, FEVER,
COUGH, SHORTNESS_OF_BREADTH,SORE_THROAT,DIARRHEA,BLUISH_LIPS_OR_FACE,
LOSS_OF_TASTE_SMELL,NAUSEA_OR_VOMITING,CONFUSED_INABILITY_AROUSE,PERSISTENT_PAIN,MUSCLE_PAIN,H
EADACHE,CONGESTION,FATIGUE,CHILLS
  FROM status_updates
  MATCH_RECOGNIZE (
  PARTITION BY person_id ORDER BY created_unified_date
  MEASURES
  person_id as person,
  HEALTH_SYSTEM_ID as HEALTH_SYSTEM_ID,
  FIRST (created_unified_date) as cluster_start,
  LAST (created_unified_date) as cluster_end,
  count (*) as NUMBER_of_updates,
  match_number ( ) as cluster_number,
  SUM
    (CASE WHEN instr (current_symptoms,‘FVR’)>0 OR instr(current_symptoms,‘CGH’)>0 OR instr
(current_symptoms,‘SOB’)>0 OR instr(current_symptoms,‘GAS’) >0 OR
instr(current_symptoms,‘TLK’) >0
  OR instr(current_symptoms,‘LTS’) >0
  THEN 1 ELSE 0 END) as NUMBER_of_days_COVID,
  SUM (CASE WHEN instr (current_symptoms,‘FVR’)>0 THEN 1 ELSE 0 END) as FEVER,
  SUM (CASE WHEN instr (current_symptoms,‘CGH’)>0 THEN 1 ELSE 0 END) as COUGH,
  SUM (CASE WHEN instr (current_symptoms,‘SOB’)>0 OR instr(current_symptoms,‘GAS’) >0 OR
instr(current_symptoms,‘TLK’) >0 THEN 1 ELSE 0 END) as SHORTNESS_OF_BREADTH,
  SUM (CASE WHEN instr (current_symptoms,‘STH’)>0 THEN 1 ELSE 0 END) as SORE_THROAT,
  SUM (CASE WHEN instr (current_symptoms,‘DIA’)>0 THEN 1 ELSE 0 END) as DIARRHEA,
  SUM (CASE WHEN instr (current_symptoms,‘BLU’)>0 THEN 1 ELSE 0 END) as BLUISH_LIPS_OR_FACE,
  SUM (CASE WHEN instr (current_symptoms,‘LTS’)>0 THEN 1 ELSE 0 END) as LOSS_OF_TASTE_SMELL,
  SUM (CASE WHEN instr (current_symptoms,‘NAU’)>0 THEN 1 ELSE 0 END) as NAUSEA_OR_VOMITING,
  SUM (CASE WHEN instr (current_symptoms,‘CIA’)>0 THEN 1 ELSE 0 END) as
CONFUSED_INABILITY_AROUSE,
  SUM (CASE WHEN instr (current_symptoms,‘PAI’)>0 THEN 1 ELSE 0 END) as PERSISTENT_PAIN,
  SUM (CASE WHEN instr (current_symptoms,‘MP’)>0 THEN 1 ELSE 0 END) as MUSCLE_PAIN,
  SUM (CASE WHEN instr (current_symptoms,‘HA’)>0 THEN 1 ELSE 0 END) as HEADACHE,
  SUM (CASE WHEN instr (current_symptoms,‘CON’)>0 THEN 1 ELSE 0 END) as CONGESTION,
  SUM (CASE WHEN instr (current_symptoms,‘FTG’)>0 THEN 1 ELSE 0 END) as FATIGUE,
  SUM (CASE WHEN instr (current_symptoms,‘CH’)>0 THEN 1 ELSE 0 END) as CHILLS
  PATTERN
    (b+)
  DEFINE
  b AS created_unified_date < prev(created_unified_date+5) OR created_unified_date = FIRST
(created_unified_date)
  )
  ) participant_clusters join people on participant_clusters.person = people.id
  ;
;
```

### Example (MATCH_RECOGNIZE) Query 1

In accordance with an embodiment, the determination of participant clusters can be used, for example, to identify continuous sets of symptom reports; or segment a population into irregular reporters versus regular reporters, and correlate their reports to actual symptoms. An identification of such participant clusters is useful in aggregating participant behavioral information at a cluster level, and correlating the information provided with actual symptoms and symptom density.

Determination of Episodes Indicative of Possible Illness

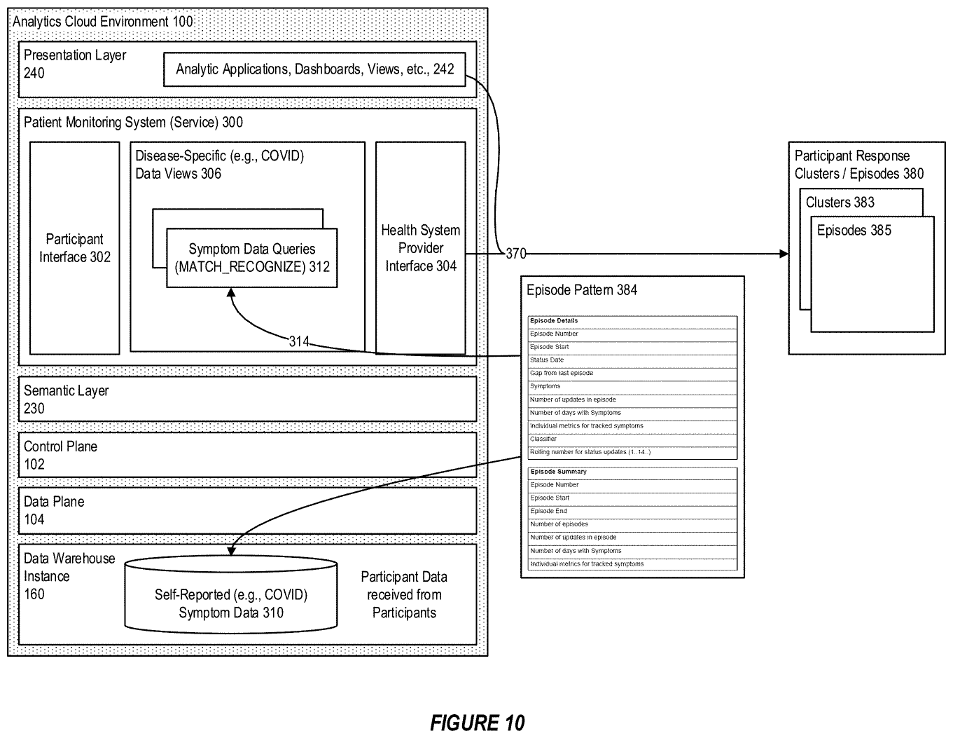

FIG. 10 further illustrates the use of a patient monitoring system or service, and pattern detection in assessing self-reported participant symptom data, to determine episodes indicative of possible illness, in accordance with an embodiment.

For example, as illustrated in FIG. 10, in accordance with an embodiment, the system can perform data queries against the received participant symptom data, as defined by an episode pattern 384, to determine episodes 385 indicative of possible illness, which information can then be provided, for example, to a medical organization system and used to respond to investigative queries.

For example, as illustrated in FIG. 10 (and in example patterns 3 and 4 below) episodes can be determined as a group of symptoms from a participant triggered by specific symptoms. The system can tracks a participant for 14 days since trigger, or longer if the symptoms continue. The system can then stop tracking once there is 5-day gap in symptoms. The system can generated detail grouped records by episodes including individual status updates; and an episode summary which summarizes participant level attributes across clusters.

Episode Details
Episode Number
Episode Start
Status Date
Gap from last episode
Symptoms
Number of updates in episode
Number of days with Symptoms
Individual metrics for tracked symptoms
Classifier
Rolling number for status updates (1 . . . 14 . . . )

### EXAMPLE PATTERN 3

### Episode Details

In accordance with an embodiment, the example episode pattern illustrated in FIG. 10, and in example patterns 3 and 4), is provided for purposes of illustration. In accordance with other embodiments, other episode patterns can be used.

Episode Summary
Episode Number
Episode Start
Episode End
Number of episodes
Number of updates in episode
Number of days with Symptoms
Individual metrics for tracked symptoms

### EXAMPLE PATTERN 4

### Episode Summary

In accordance with an embodiment, the system can be used to determine further investigations, for example: What is the exact logic for triggering an episode (or terminating an episode (how long, which symptoms)? What do we need summarized at episode/cluster level? Can one combine multiple event data (tests and status updates), for example:

Symptom X−Symptom Y−Test A−Symptom Z−Test B

As described above, in accordance with an embodiment, the patient monitoring system or service is adapted to perform SQL data queries that correspond to the defined pattern, including for example one or more pattern-matching SQL data queries that incorporate a MATCH_RECOGNIZE clause, for example as illustrated in example (MATCH_RECOGNIZE) query 2 below, to access the data warehouse or database that stores self-report participant symptom data and provide resultant information to the presentation layer and/or to the health system provider interface.

```
drop materialized view episode_details_view;
create materialized view episode_details_view
select EPISODE_DETAILS.PERSON_ID,EPISODE_DETAILS.HEALTH_SYSTEM_ID,people.facility_id,
people.ethnicity_id,episode_number,classifier,episode_start,status_date,gap,number_days_sympto
ms,NUMBER_of_updates,symptoms,
FEVER,COUGH,SHORTNESS_OF_BREATH,GASPING_FOR_AIR,LOSS_OF_TASTE_SMELL,NAUSEA_OR_VOMITING,DIARRHE
A,CONFUSED_INABILITY_AROUSE,SORE_THROAT,
BLUISH_LIPS_OR_FACE,PERSISTENT_PAIN,MUSCLE_PAIN,HEADACHE,CONGESTION,FATIGUE,CHILLS,status_numb
er, status_updates_COVID_view.COVID_3_DAY_YN
FROM (
  SELECT
  person as PERSON_ID,HEALTH_SYSTEM_ID1 as HEALTH_SYSTEM_ID,
  episode_number, classifier, trunc(episode_start) as episode_start, trunc(status_date) as
status_date,
  status_date- trunc(CASE WHEN previous_date< episode_start then episode_start else
nvl(previous_date,status_date) end) as gap,
  number_days_symptoms,NUMBER_of_updates,symptoms,
FEVER,COUGH,SHORTNESS_OF_BREATH,GASPING_FOR_AIR,LOSS_OF_TASTE_SMELL,NAUSEA_OR_VOMITING,DIARRHE
A,CONFUSED_INABILITY_AROUSE,SORE_THROAT,
  BLUISH_LIPS_OR_FACE,PERSISTENT_PAIN,MUSCLE_PAIN,HEADACHE,CONGESTION,FATIGUE,CHILLS,
  RANK ( ) OVER (PARTITION BY PERSON_ID,episode_number ORDER BY status_date) as status_number
  FROM status_updates
  MATCH_RECOGNIZE (
  PARTITION BY person_id ORDER BY created_unified_date
  MEASURES
  person_id as person,
  HEALTH_SYSTEM_ID as HEALTH_SYSTEM_ID1,
  FIRST (created_unified_date) as episode_start,
  LAST (created_unified_date) as status_date,
  PREV(created_unified_date) as previous_date,
  count (*) as NUMBER_of_updates,
  match_number ( ) as episode_number,
  classifier( ) as classifier,
  current_symptoms as symptoms,
  CASE WHEN instr (current_symptoms,‘FVR’)>0 THEN 1 ELSE 0 END as FEVER,
  CASE WHEN instr (current_symptoms,‘CGH’)>0 THEN 1 ELSE 0 END as COUGH,
  CASE WHEN instr (current_symptoms,‘SOB’)>0 OR instr (current_symptoms,‘GAS’)>0 OR instr
(current_symptoms,‘TLK’)>0
THEN 1 ELSE 0 END as SHORTNESS_OF_BREATH,
  CASE WHEN instr (current_symptoms,‘GAS’)>0 THEN 1 ELSE 0 END as GASPING_FOR_AIR,
  CASE WHEN instr (current_symptoms,‘LTS’)>0 THEN 1 ELSE 0 END as LOSS_OF_TASTE_SMELL,
  CASE WHEN instr (current_symptoms,‘NAU’)>0 THEN 1 ELSE 0 END as NAUSEA_OR_VOMITING,
  CASE WHEN instr (current_symptoms,‘DIA’)>0 THEN 1 ELSE 0 END as DIARRHEA,
  CASE WHEN instr (current_symptoms,‘CIA’)>0 THEN 1 ELSE 0 END as CONFUSED_INABILITY_AROUSE,
  CASE WHEN instr (current_symptoms,‘STH’)>0 THEN 1 ELSE 0 END as SORE_THROAT,
  CASE WHEN instr (current_symptoms,‘BLU’)>0 THEN 1 ELSE 0 END as BLUISH_LIPS_OR_FACE,
  CASE WHEN instr (current_symptoms,‘PAI’)>0 THEN 1 ELSE 0 END as PERSISTENT_PAIN,
  CASE WHEN instr (current_symptoms,‘MP’)>0 THEN 1 ELSE 0 END as MUSCLE_PAIN,
  CASE WHEN instr (current_symptoms,‘HA’)>0 THEN 1 ELSE 0 END as HEADACHE,
  CASE WHEN instr (current_symptoms,‘CON’)>0 THEN 1 ELSE 0 END as CONGESTION,
  CASE WHEN instr (current_symptoms,‘FTG’)>0 THEN 1 ELSE 0 END as FATIGUE,
  CASE WHEN instr (current_symptoms,‘CH’)>0 THEN 1 ELSE 0 END as CHILLS,
  CASE WHEN current_symptoms is NULL OR current_symptoms = ‘NUN’ THEN 0 ELSE 1 END as
number_days_symptoms
  ALL ROWS PER MATCH
  PATTERN
  ( initialSymptoms window14days+ beyond14days* )
  DEFINE
  -- at least two symptoms
  initialSymptoms as (
  instr (current_symptoms,‘NUN’)= 0 AND instr (current_symptoms,‘:’)> 0
AND current_symptoms is not null
  AND
  (
  -- Day 1 and 3
  (
  instr (NEXT(current_symptoms,2),‘NUN’)= 0 AND instr (NEXT(current_symptoms,2),‘:’)> 0
AND NEXT(current_symptoms,2) is not NULL
  AND created_unified_date+2 = NEXT(created_unified_date,2)
  )
  OR
  -- Day 1 and 4
  (
  instr (NEXT(current_symptoms,3),‘NUN’)= 0 AND instr (NEXT(current_symptoms,3),‘:’)> 0
AND NEXT(current_symptoms,3) is not NULL
  AND created_unified_date+3 = NEXT(created_unified_date,3)
  )
  OR
  -- Day 1,2 and 5
  (
  instr (NEXT(current_symptoms,1),‘NUN’)= 0 AND instr (NEXT(current_symptoms,1),‘:’)> 0
AND NEXT(current_symptoms,1) is not NULL
  AND created_unified_date+1 = NEXT(created_unified_date,1)
  AND
  instr (NEXT(current_symptoms,4),‘NUN’)= 0 AND instr (NEXT(current_symptoms,4),‘:’)> 0
AND NEXT(current _symptoms,4) is not NULL
  AND created_unified_date+4 = NEXT(created_unified_date,4)
  )
  )
  ),
  window14days AS created_unified_date <= first(created_unified_date+14),
  -- 1 or more symptom beyond day 14
  beyond14days as (created_unified_date > first(created_unified_date+14) AND
  (
  ( instr (current_symptoms,‘NUN’)= 0 AND current_symptoms is not null)
  OR
  ((instr (NEXT(current_symptoms,1),‘NUN’)= 0
AND NEXT(current_symptoms,1) is not NULL ) AND
NEXT(created_unified_date,1)<created_unified_date+5)
  OR
  ((instr (NEXT(current_symptoms,2),‘NUN’)= 0
AND NEXT(current_symptoms,2) is not NULL ) AND
NEXT(created_unified_date,2)<created_unified_date+5)
  OR
  ((instr (NEXT(current_symptoms,3),‘NUN’)= 0
AND NEXT(current_symptoms,3) is not NULL ) AND
NEXT(created_unified_date,3)<created_unified_date+5)
  OR
  ((instr (NEXT(current_symptoms,4),‘NUN’)= 0
AND NEXT(current_symptoms,4) is not NULL ) AND
NEXT(created_unified_date,4)<created_unified_date+5)
  ) )
  ))
  EPISODE_DETAILS LEFT OUTER JOIN status_updates_COVID_view
  ON EPISODE_DETAILS.person_id = status_updates_COVID_view.person_id
  AND EPISODE_DETAILS.STATUS_DATE = status_updates_COVID_view.created_unified_date
  join people on people.id = episode_details.person_id
;
```

### Example (MATCH_RECOGNIZE) Query 2

In accordance with an embodiment, the determination of episodes can be used, for example, to identify actual bouts of illness/sickness reflected in the fine-grain participant symptom data stream, and aggregate and report various parameters regarding those bouts of illness/sickness, for further classification. An identification of such episodes is useful in summarizing disease characteristics at a higher level which can be used, for example, to analyze the emergence of possible COVID infections or trends within the community.

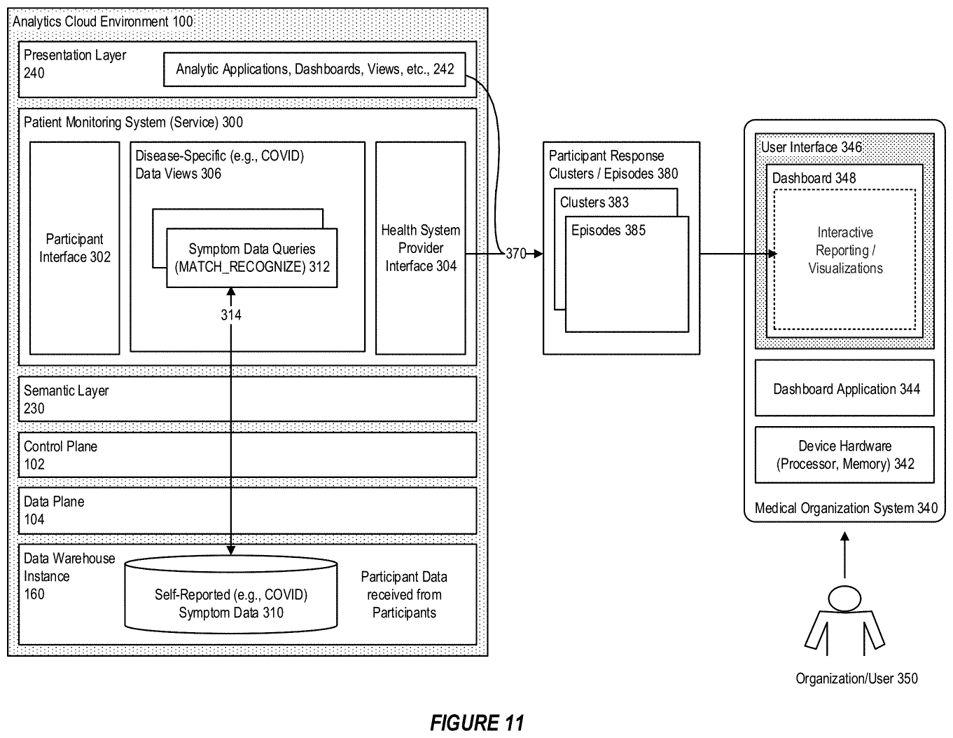

FIG. 11 further illustrates the use of a patient monitoring system or service to determine participant clusters and episodes indicative of possible illness, in accordance with an embodiment.

As illustrated in FIG. 11, information can then be provided, for example, to a medical organization system and used to respond to investigative queries. For example, in accordance with an embodiment, organizations can access de-identified participant data, and use the information to prepare data visualizations and reports.

Example Organization Dashboards

In accordance with an embodiment, the provider interface enables communication with medical organization systems or other devices that enable display of a dashboard of information that allows the organization or user to perform interactive reporting or visualizations associated with the participant symptom data.

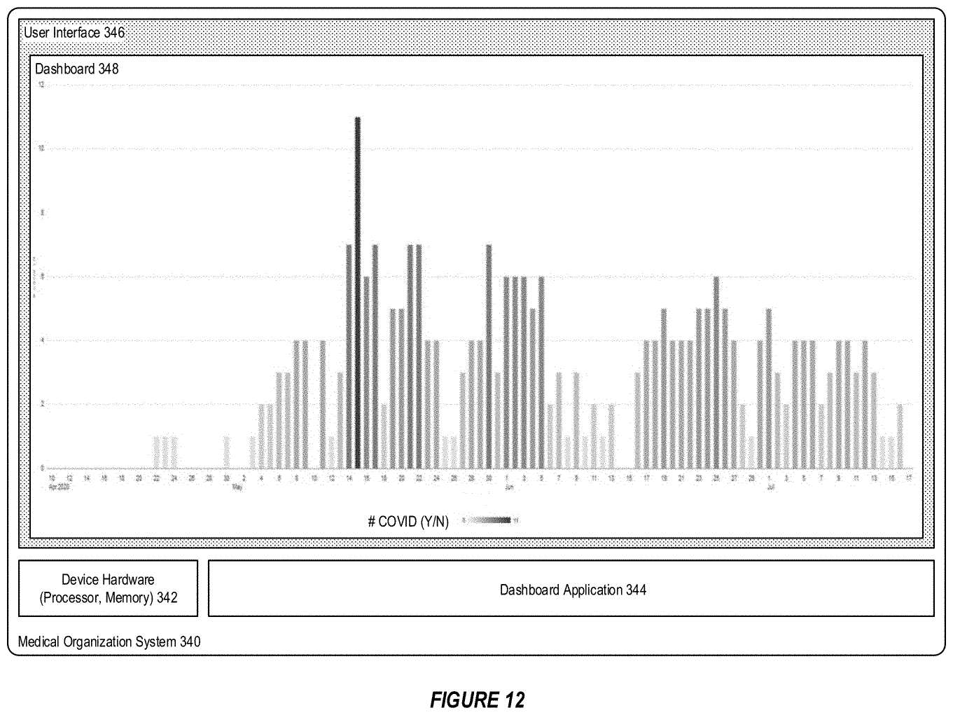

FIG. 12 illustrates an example dashboard, in accordance with an embodiment.

As illustrated in FIG. 12, in accordance with an embodiment, the system enables capture and display of self-reported participant symptom data provided by individual participants who are either suspected to have contracted, or may have been potentially exposed to, severe acute respiratory syndrome coronavirus disease (e.g., COVID), and track changes in their reported symptoms over time. Such information can then be used to identify patterns in the participant symptom data indicative of participant clusters, and episodes indicative of possible coronavirus-related illnesses.

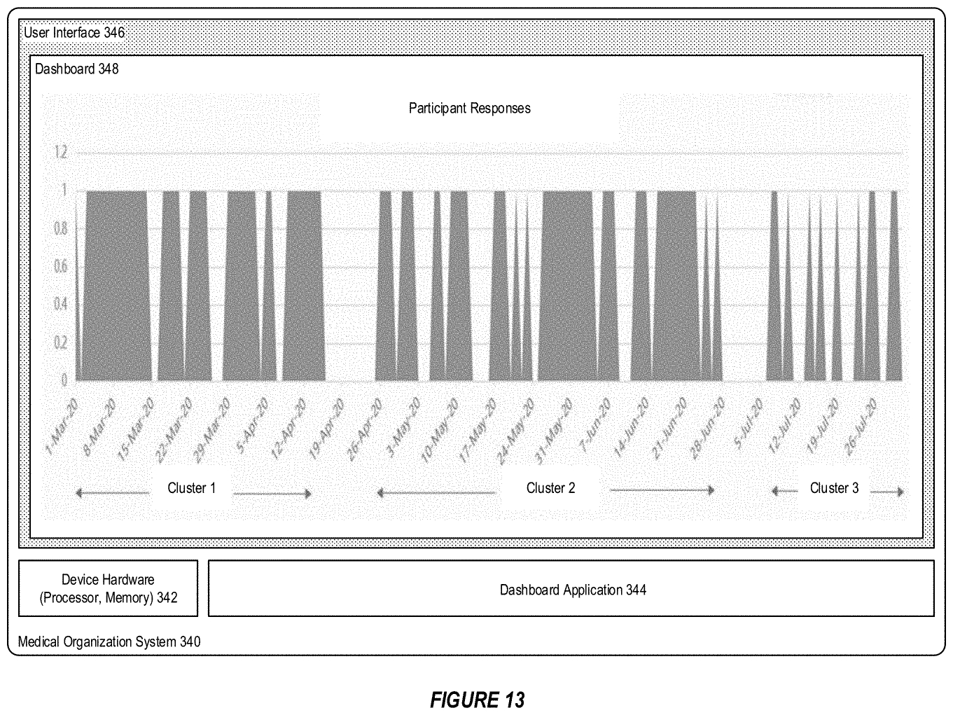

FIG. 13 illustrates another example dashboard, in accordance with an embodiment.

As illustrated in FIG. 13, in accordance with an embodiment, the determination and display of participant clusters can be used, for example, to identify continuous sets of symptom reports; or segment a population into irregular reporters versus regular reporters, and correlate their reports to actual symptoms.

For example, an identification of such participant clusters is useful in aggregating participant behavioral information at a cluster level, and correlating the information provided with actual symptoms and symptom density.

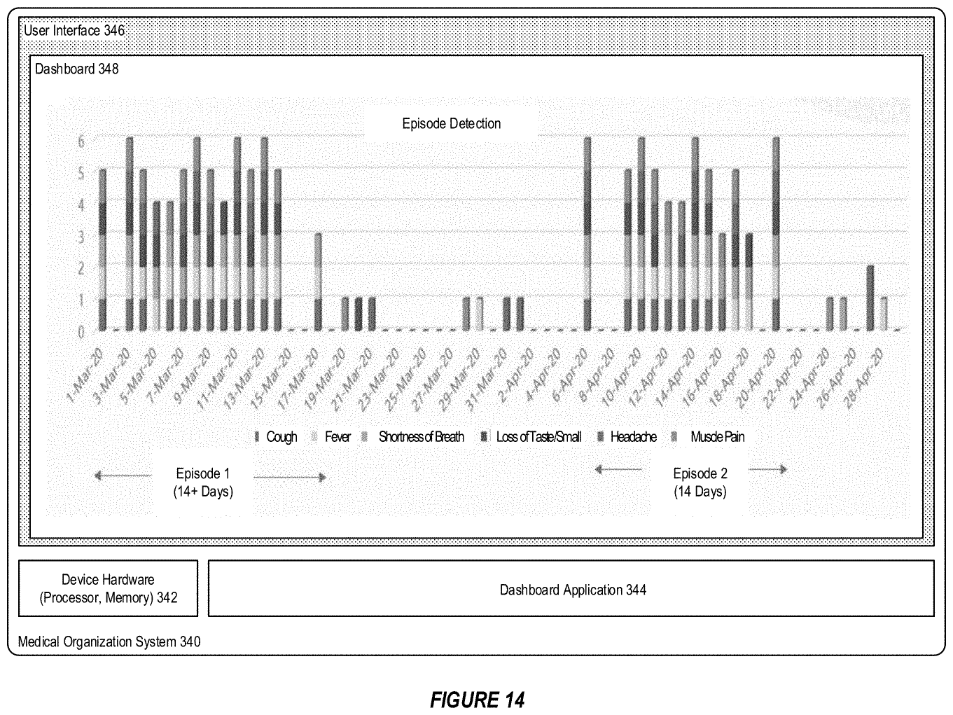

FIG. 14 illustrates another example dashboard, in accordance with an embodiment.

As illustrated in FIG. 14, in accordance with an embodiment, the determination and display of episodes can be used, for example, to identify actual bouts of illness/sickness reflected in the fine-grain participant symptom data stream, and aggregate and report various parameters regarding those bouts of illness/sickness, for further classification.

For example, an identification of such episodes is useful in summarizing disease characteristics at a higher level which can be used, for example, to analyze the emergence of possible COVID infections or trends within the community.

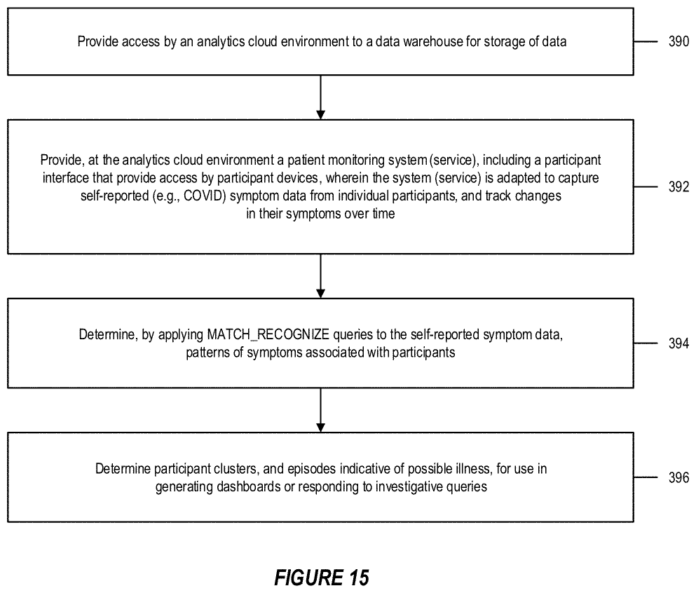

FIG. 15 illustrates an example method or process for providing a patient monitoring system or service, including support for use of pattern detection in assessing self-reported participant symptom data, in accordance with an embodiment.

As illustrated in FIG. 15, in accordance with an embodiment, at step 390. An analytics cloud environment provides access to a data warehouse for storage of data.

At step 392, a patient monitoring system (service), including a participant interface that provide access by participant devices is provided, wherein the system (service) is adapted to capture self-reported (e.g., COVID) symptom data from individual participants, and track changes in their symptoms over time.

At step 394, by applying MATCH_RECOGNIZE data queries to the self-reported participant symptom data, patterns of symptoms associates with participants can be determined.

At step 396, the system determines participant clusters, and episodes indicative of possible illness, for use in generating dashboards or responding to investigative queries.

Example Participant Surveys

In accordance with an embodiment, the systems and methods described above can be used to provide a patient monitoring system, for example at an analytics cloud environment, which can also be shared by various organizations, such as for example hospital systems, insurance providers, universities, senior living facilities, pharmacies, and workplaces, for purposes of monitoring public health during, e.g., a COVID pandemic.

For example, in accordance with an embodiment, the patient monitoring system allows organizations to track population trends for symptoms, exposure, and preventative actions related to COVID, by inviting the public to report daily activities. Organizations can access de- identified participant data, and use the information to prepare data visualizations and reports that are useful in addressing the pandemic.

In accordance with an embodiment, the participant interface enables communication with mobile devices or other devices that include a participant reporting application and that are adapted receive from participants a symptom data, for example via a survey.

For example, in accordance with an embodiment, an organization can invite individuals to report their daily symptoms, behaviors, treatments, contacts, use of personal protective equipment, or other factors. Participants receive a request to share their information via a daily update, for example by responding to an email or text survey. An organization can create one or more additional or custom surveys, such as, for example:

Standard daily: A standard daily survey.

Custom daily: A custom daily survey that allows the organization customize the questions in the daily update.

Supplemental: A supplemental survey that contains questions that participants complete in addition to the standard or custom daily survey; for example, to capture information about whether participants participated in non-household gatherings, or exercised safeguards during a holiday weekend.

In-office: An in-office survey that a healthcare practitioner completes for a participant, usually as part of an in-office or telehealth visit, and is suitable for information that is best collected from a practitioner in an office situation, or that might require medical interpretation.

On-demand: An on-demand survey that a practitioner completes for a participant after an event has occurred, and is suitable for information that is best collected from a practitioner in an office situation, or that might require medical interpretation.

In accordance with an embodiment, an organization can also create surveys at various levels, such as, for example: Organization: for all participants in the organization; or Entity: for all participants in one entity. An organization can also determine the frequency of custom surveys, for example, to send the custom daily survey every other day rather than every day, or to start on a particular date.

The above feature are provided by way of example, to illustrated a particular embodiment or example of the types of features and surveys that can be supported by a patient monitoring system. In accordance with other embodiments, other types of features and surveys that can be supported by the patient monitoring system.

In accordance with various embodiments, the teachings herein may be conveniently implemented using one or more conventional general purpose or specialized computer, computing device, machine, or microprocessor, including one or more processors, memory and/or computer readable storage media programmed according to the teachings of the present disclosure. Appropriate software coding can readily be prepared by skilled programmers based on the teachings of the present disclosure, as will be apparent to those skilled in the software art.

In some embodiments, the teachings herein can include a computer program product which is a non-transitory computer readable storage medium (media) having instructions stored thereon/in which can be used to program a computer to perform any of the processes of the present teachings. Examples of such storage mediums can include, but are not limited to, hard disk drives, hard disks, hard drives, fixed disks, or other electromechanical data storage devices, floppy disks, optical discs, DVD, CD-ROMs, microdrive, and magneto-optical disks, ROMs, RAMs, EPROMs, EEPROMs, DRAMs, VRAMs, flash memory devices, magnetic or optical cards, nanosystems, or other types of storage media or devices suitable for non-transitory storage of instructions and/or data.

The foregoing description has been provided for the purposes of illustration and description. It is not intended to be exhaustive or to limit the scope of protection to the precise forms disclosed. Many modifications and variations will be apparent to the practitioner skilled in the art.

The embodiments were chosen and described in order to best explain the principles of the present teachings and their practical application, thereby enabling others skilled in the art to understand the various embodiments and with various modifications that are suited to the particular use contemplated. It is intended that the scope be defined by the following claims and their equivalents.

What is claimed is:

1. A system for use of data analytics in medical applications, including the use of pattern detection in assessing self-reported participant symptom data indicative of possible illness, comprising:
a computing environment comprising a processor and providing access to a data warehouse for storage of a participant symptom data; and
a patient monitoring service provided at the computing environment and adapted to capture self-reported participant symptom data from individual participants, and track changes in their reported symptoms over time; and
wherein the system performs data queries against the received participant symptom data, to identify patterns in the data indicative of participant clusters and episodes indicative of possible illness, which information can then be used to respond to investigative queries.

2. The system of claim 1, wherein the patient monitoring service is provided at a cloud environment and accessed as a service via one or more participant or provider interfaces.

3. The system of claim 1, wherein the data queries include MATCH_RECOGNIZE data queries that the system performs against the received participant symptom data, to identify patterns in the data.

4. The system of claim 1, wherein a participant interface enables communication with mobile devices or other devices that include a participant reporting application and that are adapted receive from participants a symptom data via a survey.

5. A method for use of data analytics in medical applications, including the use of pattern detection in assessing self-reported participant symptom data indicative of possible illness, comprising:
providing access by a computing environment to a data warehouse for storage of a participant symptom data;
providing a patient monitoring service provided at the computing environment and adapted to capture self-reported participant symptom data from individual participants, and track changes in their reported symptoms over time; and
performing data queries against the received participant symptom data, to identify patterns in the data indicative of participant clusters and episodes indicative of possible illness, which information can then be used to respond to investigative queries.

6. The method of claim 5, wherein the patient monitoring service is provided at a cloud environment and accessed as a service via one or more participant or provider interfaces.

7. The method of claim 5, wherein the data queries include MATCH_RECOGNIZE data queries that the system performs against the received participant symptom data, to identify patterns in the data.

8. The method of claim 5, wherein a participant interface enables communication with mobile devices or other devices that include a participant reporting application and that are adapted receive from participants a symptom data via a survey.

9. A non-transitory computer readable storage medium, including instructions stored thereon which when read and executed by one or more computers cause the one or more computers to perform a method comprising:
providing access by a computing environment to a data warehouse for storage of a participant symptom data;
providing a patient monitoring service provided at the computing environment and adapted to capture self-reported participant symptom data from individual participants, and track changes in their reported symptoms over time; and
performing data queries against the received participant symptom data, to identify patterns in the data indicative of participant clusters and episodes indicative of possible illness, which information can then be used to respond to investigative queries.

10. The non-transitory computer readable storage medium of claim 9, wherein the patient monitoring service is provided at a cloud environment and accessed as a service via one or more participant or provider interfaces.

11. The non-transitory computer readable storage medium of claim 9, wherein the data queries include MATCH_RECOGNIZE data queries that the system performs against the received participant symptom data, to identify patterns in the data.

12. The non-transitory computer readable storage medium of claim 9, wherein a participant interface enables communication with mobile devices or other devices that include a participant reporting application and that are adapted receive from participants a symptom data via a survey.
